(19) United States
(12) Patent Application Publication
(10) Pub. No.: US 2019/0066832 A1
(43) Pub. Date: Feb. 28, 2019

(54) METHOD FOR DETECTING PATIENT RISK AND SELECTIVELY NOTIFYING A CARE PROVIDER OF AT-RISK PATIENTS

(71) Applicant: KangarooHealth, Inc., San Jose, CA (US)

(72) Inventors: Xiaoxu Kang, San Jose, CA (US); Kevin Olds, Baltimore, MD (US)

(21) Appl. No.: 15/895,981

(22) Filed: Feb. 13, 2018

Related U.S. Application Data
(60) Provisional application No. 62/461,157, filed on Feb. 20, 2017.

(51) Int. Cl. G16H 20/30; A61B 5/00; A61B 5/11; G16H 10/60

(52) U.S. Cl. CPC G16H 20/30; A61B 5/0077; A61B 5/6898; A61B 5/7275; A61B 5/4528; A61B 5/1121; A61B 2505/09; A61B 5/4884; A61B 5/4878; A61B 5/7278; A61B 5/0022; G16H 10/60; A61B 5/1128

(57) ABSTRACT

One variation of a method for tracking patient recovery during a physical therapy program includes: assigning a recovery plan to a patient, the recovery plan defining temporal quantitative targets for range of motion of a joint; during a first physical therapy session: prompting the patient to record a first digital photographic image of the joint in flexion; and prompting the patient to record a second digital photographic image of the joint in extension. The method also includes extracting an angular range of motion of the joint from the first digital photographic image and the second digital photographic image; in response to the angular range of motion of the joint deviating from the recovery plan by more than a threshold deviation, compiling the angular range of motion of the joint into a notification; and serving the notification to a computing device associated with a care provider.

### CROSS-REFERENCE TO RELATED APPLICATIONS

This Application claims the benefit of U.S. Provisional Application No. 62/461,157, filed on 20 Feb. 2017, which is incorporated in its entirety by this reference.

### TECHNICAL FIELD

This invention relates generally to the field of patient recovery and more specifically to a new and useful method for detecting patient risk and selectively notifying a care provider of at-risk patients in the field of patient recovery.

### BRIEF DESCRIPTION OF THE FIGURES

FIGS. 1A and 1B are flowchart representations of a method;

FIG. 2 is a flowchart representation of a variation of the method;

FIG. 3 is a flowchart representation of a variation of the method; and

FIG. 4 is a flowchart representation of a variation of the method.

### DESCRIPTION OF THE EMBODIMENTS

The following description of embodiments of the invention is not intended to limit the invention to these embodiments but rather to enable a person skilled in the art to make and use this invention. Variations, configurations, implementations, example implementations, and examples described herein are optional and are not exclusive to the variations, configurations, implementations, example implementations, and examples they describe. The invention described herein can include any and all permutations of these variations, configurations, implementations, example implementations, and examples.

### 1. Method

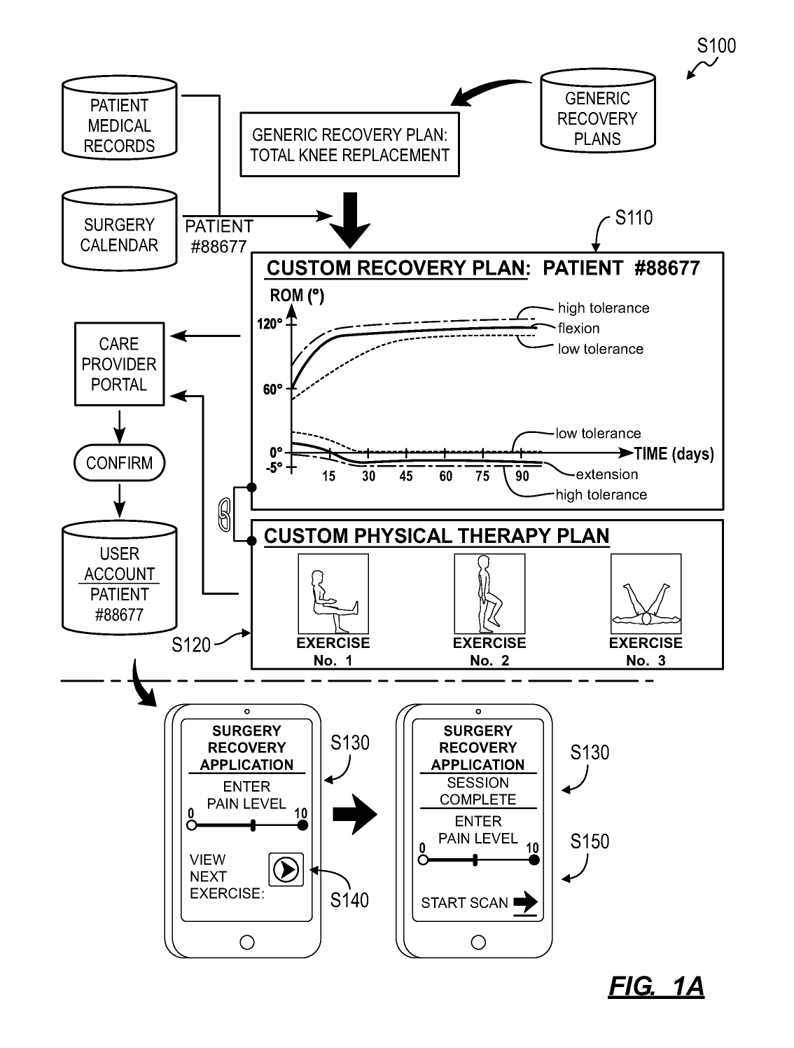

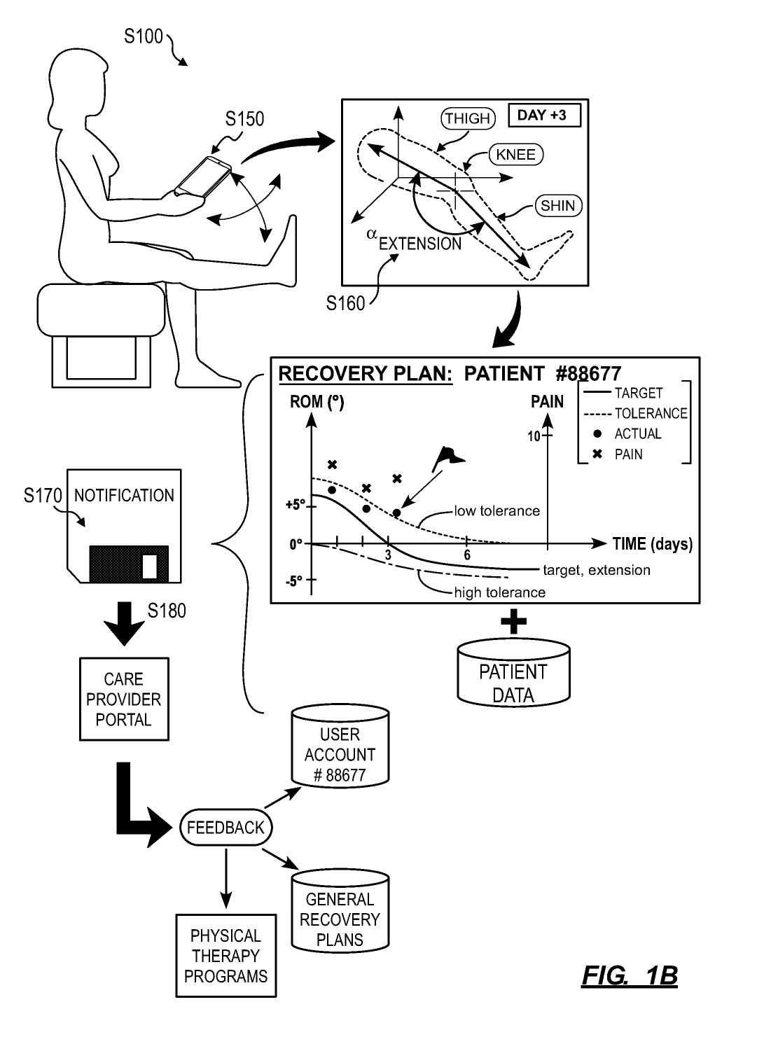

As shown in FIGS. 1A and 1B, a method S100 for detecting patient risk and selectively notifying a care provider of at-risk patients during a physical therapy program includes: assigning a recovery plan to a patient, the recovery plan defining temporal quantitative targets for range of motion of a joint in Block S110. Additionally, the method S100 includes, at a mobile computing device during a first physical therapy session: prompting the patient to record a first digital photographic image of the joint in flexion in Block S150; and prompting the patient to record a second digital photographic image of the joint in extension in Block S152. The method S100 further includes: extracting a first angular range of motion of the joint from the first digital photographic image and the second digital photographic image in Block S160; in response to the first angular range of motion of the joint deviating from the recovery plan by more than a threshold deviation, compiling the first angular range of motion of the joint and an identifier of the patient into a notification in Block S170; and serving the notification to a computing device associated with a care provider in Block S180.

One variation of the method S100 includes, at the mobile computing device during the first physical therapy session: prompting the patient to position the mobile computing device at a first position on a first side of the joint in extension in Block S154; prompting the patient to position the mobile computing device at a second position on a second side of the joint in extension, the second side opposite the joint from the first side of the joint in Block S155; prompting the patient to position the mobile computing device at a third position on the first side of the joint in flexion in Block S157; and prompting the patient to position the mobile computing device at a fourth position on the second side of the joint in flexion in Block S158. This variation of the method S100 also includes: extracting a first angular range of motion of the joint as a difference between an angle of extension and an angle of flexion, the angle of extension defined between the first orientation and the second orientation, the angle of flexion defined between the third orientation and fourth orientation in Block S162.

Another variation of the method S100 further includes: assigning a recovery plan to a patient based on a surgery prescribed to the patient in Block S110, the recovery plan defining temporal quantitative targets for range of motion of a joint addressed in the surgery; and assigning a physical therapy program to a patient in Block S120, the physical therapy program defining a set of physical therapy exercises; at the mobile computing device, during a first physical therapy session, prompting the patient to enter a first pain level in Block S130, serving a first physical therapy exercise—in the physical therapy program—to the patient in Block S140.

### 2. Applications

Generally, the method can be implemented on behalf of a patient during a physical therapy program (e.g., following a surgery) to: define quantitative target metrics for the patient's recovery; to extract quantitative metrics of the patient's actual recovery from scans of an area of interest on the patient's body; and to selectively involve a care provider—such as a surgeon, a nurse, or physical therapist—if quantitative metrics of the patient's actual recovery differ significantly from the quantitative target metrics. In particular, Blocks of the method can be executed by a local or remote computing device (e.g., the patient's mobile computing device, such as a smartphone, in cooperation with a remote server) to: assign temporal quantitative target metrics for post-operative recovery to a patient; assign physical therapy exercises—that the patient may perform on her own at home—to the patient; guide the patient in recording a 2D or 3D scan (e.g., optical images and/or contour maps of the patient's joint collected at a mobile computing device before, during, and/or after a physical therapy session) of a region of her body, such as her knee following a total knee replacement surgery; extract range of motion values in this region of the patient's body (e.g., ranges of motion of the patient's knee in flexion and extension) from these scans; track the patient's recovery based on these range of motion values; and selectively prompt a care provider to intervene in the patient's recovery if the range of motion values in this region of the patient's body do not sufficiently align with the temporal quantitative target metrics assigned to her.

By assigning quantitative target recovery metrics to the patient and collecting quantitative data from the patient during her recovery, a system executing the method can define a measurable standard of care for post-operative recovery of the patient. The system can also: guide a patient through prescribed post-operative exercises; automatically track the patient's recovery over time without the need for involvement of another human by analyzing optical scans of the patient's body; and selectively prompt human care providers to address the patient's recovery when certain quantitative triggers are met, such as if the patient's pain level is trending upward, if the patient is failing to comply with the physical therapy program or optical scan schedule, or if the range of motion of the patient's joint of interest is not meeting generic or custom quantitative target metrics assigned to the patient. Therefore, by implementing Blocks of the method, the system can provide flexibility to a patient in performing post-operative physical therapy exercises, such as at home or work and without the assistance of a physical therapist. Thus, the system can enable a patient autonomy over her recovery and completion of her assigned physical therapy regimen by guiding the user through automated therapy sessions and only involving a care provider (e.g., physical therapy and/or a doctor) in the patient's recovery when the patient exhibits symptoms and/or trends indicating deviation from her recovery plan.

Furthermore, the system can: rapidly detect deviations—even minor deviations—in a patient's recovery from a predicted recovery path; calculate risk to the patient (e.g., infection, poor range of motion, risk of complications) based on range of motion of a joint of interest (or wound images or pain scores) in the patient; and selectively involve care providers when calculated risk to the patient exceeds a preset threshold. Therefore, the system can function to improve efficiency and efficacy of care providers in supporting patients by identifying high-risk patients, distinguishing lower-risk patients, and selectively prompting involvement of care providers (e.g., nurses, physical therapists, doctors, surgeons, or family members) with high-risk patients at opportunistic times.

### 2.1 EXAMPLES

The method is described herein as implemented in conjunction with a physical therapy program prescribed to a patient following a total knee replacement surgery. For example, in preparation for a total knee replacement surgery in a patient's left knee, the patient can install or load a native application and/or other window rendering a patient portal executing on her mobile computing device (e.g., a smartphone), such as responsive to advice provided to the patient by the patient's doctor or nurse, and create a patient account within the patient portal. (Alternatively, the patient's doctor, nurse, or technician can provide the patient a mobile computing device (e.g., a tablet) preloaded with an instance of a patient portal, such as before the surgery and while teaching the patient proper use of the patient portal.) The patient's doctor, nurse, or physical therapist can assign a recovery plan and a physical therapy program to the patient's account. Following the surgery, the patient can access the patient portal on her mobile computing device regularly (e.g., once daily) to: indicate perceived pain level in her left knee; view prescribed physical therapy exercises, such as in the form of narrated videos and/or textual exercise, time, and repetition descriptions; and to manually scan her left knee with an optical sensor (e.g., a color camera) integrated into the mobile computing device. The patient can then process a scan of the patient's left knee locally in Block S150 to determine range of motion in the left knee. (Alternatively, the patient portal can return scan data to a remote server, which can process these scan data remotely.) If the patient (or the remote computer system) calculates a range of motion in the patient's left knee that deviates from the assigned recovery plan at a single instant in time or that is trending away from the assigned recovery plan, the patient (or the remote computer system) can notify a care provider associated with the patient in Blocks S170 and S180.

However, Blocks of the method can be implemented in conjunction with a physical therapy program, such as prescribed to a patient following any other surgery, such as: a hip replacement surgery; a shoulder arthroscopy surgery; a carpal tunnel release surgery; an anterior cruciate ligament (“ACL”) reconstruction surgery; surgical repair of a femoral neck fracture; a lumbar spinal fusion surgery; surgical repair of an incise finger tendon sheath; and/or surgical repair of a femoral shaft fracture; etc. Furthermore, Blocks of the method can be implemented in conjunction with a physical therapy program prescribed to a patient outside of post-operative recovery, such as following a sport-related or work-related injury or fall.

### 3. System

Blocks of the method are described below as executed by a system, such as including: a patient portal (and/or portal rendered within a web-browser) executing on a mobile computing device associated with or assigned to the patient; a remote computer system; a care provider portal accessible by a doctor, nurse, technician, and/or physical therapist; and/or a medical record database, etc.

For example, the system can include a mobile computing device affiliated with the patient and configured to record photographic images. The mobile computing device can include an inertial measurement unit (or “IMU”), such as an accelerometer, gyroscopic sensor, and/or compass sensor, configured to detect geospatial orientation of the mobile computing device. In this example, the mobile computing device can render a patient portal, such as through a web-browser and/or a native application, configured to: prompt the patient to record digital photographic images of the joint in extension and flexion in Block S150 and upload digital photographic images of the joint to a remote computer system and/or server. The remote computer system can then execute Blocks of the method to extract the angular range of motion and selectively prompt a care provider to contact a patient in response to detecting a deviation from a recovery plan as described below.

Additionally or alternatively, the system can record photographic images at the mobile computing device, locally anonymize the photographic images to remove patient-specific information from the images at the mobile computing device, and upload the images to a remote computer system.

Furthermore, the system can execute Blocks of the method S100 locally at the mobile computing device to detect deviations from the recovery plan and transmit notifications of deviations from the recovery plan to a care provider portal rendered on a computing device affiliated with a care provider.

### 4. Recovery Plan

Block S110 of the method recites assigning a recovery plan to a patient based on a surgery prescribed to the patient, wherein the recovery plan defines temporal quantitative targets for range of motion of a joint addressed in the surgery (e.g., a “joint of interest”). Generally, in Block S110, the system accesses a recovery plan that defines quantitative targets for range of motion in a joint of interest over time and assigns this recovery plan to the patient as a reference for monitoring the patient's recovery following surgery on or near the joint of the interest.

In particular, a surgical operation on bone, cartilage, ligament, or muscle tissue, etc. at or near a joint may result in post-operative inflammation, stiffness, and weakness in and around the joint and reduced range of motion (e.g., in flexion, extension, adduction, abduction, and rotation) at the joint. However, increases in range of motion of the joint over time may indicate healing in and around the joint and may suggest an eventual outcome of the surgery, such as whether the patient achieves a full recovery with a return to full range of motion of the joint or whether further complications will arise. By assigning a recovery plan to the patient following a surgical operation at or around a joint of interest, the system can specify target ranges of motion (or discrete target angles in flexion, extension, adduction, abduction, and/or rotation, etc.) for the patient's joint of interest over time; by comparing these target ranges of motion to actual ranges of motion of the joint of interest—extracted from optical scans of the patient's body in Block S150 and/or extracted from contact-based scans in Block S156 and S158—the system can determine whether the patient's recovery is deviating from a normal or anticipated trajectory in Block S160 and then predict complications in the patient's recovery and notify a care provider accordingly in Block S170.

In one example shown in FIGS. 1A and 1B, the recovery plan includes: a flexion curve representing a target joint angle in flexion—in degrees—of the joint over a period of time (e.g., 90 days); and a low tolerance curve offset (or lower “threshold deviation”) from the flexion curve and representing a cumulative allowable shortage from the flexion curve over the period of time. For a joint for which limitations on the range of motion of the joint post-operation is desired, such as to ensure proper healing of adjacent muscle tissue. The recovery plan can also include a high tolerance curve offset (or upper “threshold deviation”) from the flexion curve and representing a cumulative allowable overage over the flexion curve throughout the period of time. In this example, the flexion curve (and the tolerance curve(s)) can define a smooth, continuous curve defining target joint angles over the period of time. Alternatively, the flexion curve can define one target joint angle value per day (or per week or other time interval).

In the foregoing example, the recovery plan can similarly include: an extension curve representing a target joint angle in extension of the joint over the same period of time; a low tolerance curve offset from the extension curve and representing a cumulative allowable shortage from the extension curve over the period of time; and/or a high tolerance curve offset (or “threshold deviation”) from the extension curve and representing a cumulative allowable overage over the extension curve throughout the period of time. For the joint that also exhibits motion in adduction internal rotation, and/or inversion, the recovery plan can further include: target joint angle, low tolerance and/or high tolerance curves for adduction, abduction, internal rotation, external rotation, inversion, and/or eversion of the joint over the same period of time.

However, the recovery plan can include any other number of curves, each defining a target joint angle for range of motion of the joint in a particular degree of freedom of the joint.

Alternatively, the recovery plan can contain the foregoing data represented in the form of a lookup table, plot, or parametric model or in any other form.

### 4.1 Generic Recovery Plan

In one implementation, the system automatically assigns a preset, static recovery plan to the patient based on the type of surgery prescribed to the patient and/or type of injury of the patient (e.g., lower extremity pain, ligament tear, fracture, etc.). For example, the system can automatically: retrieve a type and date of an upcoming or recent surgery scheduled for the patient (e.g., from the patient's medical record); retrieve a copy of a preset, static recovery plan specific to the surgery type and previously defined by a hospital, surgical group, health insurance agency, physical therapy group, or other entity affiliated with the patient; inject the surgery date into the copy of the recovery plan to locate target range of motion values in time; and then write the recovery plan to the patient's user account.

In one example, for a total knee replacement surgery, the generic recovery plan can include a flexion curve and corresponding tolerance curves that specify: a target angle of 80°±10° in flexion at the knee at day-three following the surgery; a target angle of 110°±10° in flexion at the knee at two weeks following the surgery; and a minimum of 100° in flexion at the knee for each subsequent week up to 90 days following the surgery. In this example, the recovery plan for the total knee replacement surgery can also include an extension curve and corresponding tolerance curves that specify: a target angle of 5°±5° in flexion at the knee at day-three following the surgery; and a maximum of 0° in flexion at the knee for each subsequent week up to 90 days following the surgery. For a patient prescribed or who has recently undergone a total knee replacement surgery, the system can automatically assign this generic recovery plan to the patient and load the generic recovery plan into the patient's user account.

Alternatively, a care provider affiliated with the patient can manually select the static recovery plan for the patient and enter a start date for the static recovery plan, and the system can write the static recovery plan to the patient's account once confirmed by the care provider. Furthermore, a care provider affiliated with the patient can manually define the static recovery plan for the patient and write the static recovery plan to the patient's account through a care provider portal.

### 4.2 Custom Recovery Plan

Alternatively, the system can write a customized recovery plan to the patient's user account, such as automatically or under supervision of a care provider. In one implementation, the system automatically selects a generic recovery plan for the surgery type prescribed to the patient and then prompts a physical therapist (or other care provider) to customize the recovery plan for the patient through a care provider portal. For example, if the patient exhibited low range of motion in her left knee, such as limited to 5° in extension to 80° in flexion, such as indicated in a preoperative chart or medical record for the patient, the physical therapist can reduce the long-term target extension and flexion angles to 0° and 90° (from −5° to 110°) for the patient, and the system can scale flexion, extension, and tolerance curves in the recovery plan accordingly and write this custom recovery plan to the patient's user account. In another example, the physical therapist can set reduced long-term target extension and flexion angles for older patients and/or increase long-term target extension and flexion angles for younger patients; and the system can scale flexion, extension, and tolerance curves in recovery plans assigned to patients accordingly.

In another implementation, the system: accesses a parametric recovery model specific to the surgery type prescribed to the patient; extracts various data from the patient's medical record, such as preoperative range of motion in the joint of interest, weight or body-mass index, age, gender, and/or preoperative activity level or mobility; passes these data into the parametric recovery model to generate a custom recovery plan for the patient; and writes the custom recovery plan to the patient's user account. For example, the parametric recovery model can output a recovery plan specifying greater range of motion targets, looser low tolerance curves, and tighter high tolerance curves for younger patients exhibiting greater preoperative range of motion, lower weight or body-mass index, and/or greater preoperative mobility. In this example, the parametric recovery model can similarly output a recovery plan specifying lower range of motion targets, tighter low tolerance curves, and looser high tolerance curves for older patients exhibiting lesser preoperative range of motion, greater weight or body-mass index, and/or lower preoperative mobility. In this implementation, the system can also prompt a care provider, such as the patient's assigned physical therapist, to review and manually adjust daily or overall range of motion curves in the custom recovery plan prior to writing the custom recovery plan to the patient's user account.

However, the system can assign a generic or custom recovery plan to the patient automatically or in cooperation with a care provider in any other way and according to any other parameters in Block S110.

### 5. Physical Therapy Program

One variation of the method S100 includes assigning a physical therapy program to a patient, wherein the physical therapy program defines a set of physical therapy exercises in Block S120. Generally, in Block S120, the system writes a physical therapy program—specifying one or more physical therapy exercises and an exercise schedule for aiding recovery from a surgery—to the patient's user account; the patient can then access information related to these physical therapy exercises and the exercise schedule to perform these exercises independently and outside of a clinic setting, such as at home or at work.

In one implementation, the system retrieves a copy of a generic physical therapy program associated with a particular surgery type prescribed to or recently completed by the patient and assigns this generic physical therapy program to the patient's user account. For example, a generic physical therapy program can include: a default list of unique physical therapy exercises designed to reduce inflammation, improve strength, and increase range of motion in a joint of interest following a particular type of surgery; a default physical therapy schedule specifying which physical therapy exercises the patient is to complete during physical therapy sessions on specific days following a surgery on or around the joint of interest and a default intensity level or default number of repetitions (or sets or duration of exercise) for each physical therapy exercise to be performed during each physical therapy session; and a set of videos (or images and/or audio cues) exhibiting proper technique for each physical therapy exercise in the list. Following the surgery, the patient can thus access the physical therapy schedule and view videos of physical therapy exercises through a native application and/or the patient portal executing on her mobile computing device and perform these exercises, as described below.

In a similar implementation, the system can adjust intensity levels and/or numbers of repetitions specified for each physical therapy exercise during each post-operative physical therapy session—such as automatically or under guidance from a care provider—based on various patient data. For example, the system (or the care provider) can increase intensity levels and/or number of repetitions specified for physical therapy exercises during post-operative physical therapy sessions: directly proportional to the patient's preoperative range of motion in the joint of interest and preoperative activity level or mobility; and inversely proportional to the patient's weight, body-mass index, and/or age. In a similar implementation, the system can automatically adjust intensity levels and/or number of repetitions specified for physical therapy exercises assigned to the patient to match the recovery plan assigned to the patient in Block S110. For example, the system can automatically write more aggressive intensity levels and/or number of repetitions for various physical therapy exercises in the physical therapy program to correspond to a more aggressive recovery plan assigned to the patient in Block S110, and vice versa.

The system can implement similar methods and techniques to automatically add or remove physical therapy exercises from all or select physical therapy sessions prescribed in the physical therapy program in order to match the physical therapy program to the recovery plan assigned to the patient. Additionally or alternatively, the system can adjust the physical therapy program pre-operatively (and prior to commencement of the program) to adapt to pre-operative scans and/or patient data input by the patient and/or care provider as described below.

In another implementation, in Block S110, the system accesses a database of unique recovery plan templates and automatically selects a particular recovery plan template associated with patient parameters (e.g., preoperative range of motion in the joint of interest, weight or body-mass index, age, gender, and/or preoperative activity level or mobility, etc.) that best matches like parameters of the patient. In Block S120, the system can then retrieve a physical therapy program specifically associated with (e.g., matched to) the particular recovery plan template selected for the patient. The system can therefore selectively assign a predefined recovery plan and a matched physical therapy program to the patient based on the type of surgery prescribed to the patient and various patient data. In particular, in this implementation, the system can implement various patient parameters to match the patient to a particular recovery plan in Block S110 and then select a corresponding physical therapy program defining physical therapy exercises predicted to enable the patient to achieve target range of motion metrics on specific days post-operation, as defined in the particular recovery plan, if followed by the patient.

In the foregoing implementations, the system can also prompt the care provider to manually adjust physical therapy exercises and corresponding intensity levels and/or repetitions defined in the physical therapy program specifically for the patient through the care provider portal. In particular, the system can enable the care provider to manually customize various aspects of the physical therapy program prior to writing the physical therapy program to the patient's user account.

### 6. Physical Therapy Session

Blocks S130 and S140 of the method recite: at a mobile computing device, prompting the patient to enter a first pain level during a first physical therapy session; and serving a first physical therapy exercise, in the physical therapy program, to the patient, respectively. Generally, the system collects pain perception data from the patient in Block S130 and serves physical therapy exercise information to the patient in Block S140, such as through the patient portal executing on the patient's mobile computing device.

In one implementation, the patient portal automatically serves a prompt to the patient to enter her perceived pre-exercise pain level (e.g., when the patient portal is opened by the patient). For example, the patient portal can prompt the patient to enter a number between 0 and 10, to move a slider between “no pain” and “severe pain” positions, or speak a quantitative of qualitative pain level into the mobile computing device; and the patient portal can record the patient's pain level entry locally and/or upload this value to a remote computer system for processing.

Once the pain level is entered by the patient, the computer system can automatically display physical therapy exercise information to the patient, such as by displaying a list of physical therapy exercises and corresponding intensity levels and/or repetition targets prescribed to the patient for the current day or current physical therapy session by the physical therapy program. The patient portal can also display thumbnail images of videos showing these physical therapy exercises and can replay these videos for the patient upon selection of the corresponding thumbnail image. For example: the patient portal can serve a textual description and a descriptive video of a first physical therapy exercise to the patient. The patient can then perform this first physical therapy exercise and confirm within the patient portal that this first physical therapy exercise was completed; the patient portal can then serve a textual description and a descriptive video of a second physical therapy exercise to the patient; the patient can perform this second physical therapy exercise and confirm within the patient portal that this second physical therapy exercise was completed; and the patient portal and the patient can repeat this process until all physical therapy exercises for the current physical therapy session are completed.

Once the physical therapy exercises for the current physical therapy session are completed, the patient portal can implement methods and techniques described above to prompt the patient to enter her perceived post-exercise pain level.

In the foregoing implementation, the patient portal can alternatively actively push a prompt to enter a pre-exercise pain level and/or push physical therapy exercise information to the patient, such as through a home screen or locked-screen notification, at preset physical therapy session times in order to actively prompt the patient to manage her post-operative recovery by performing an physical therapy exercise, completing a survey (e.g., a pain level survey), and scanning her joint of interest.

However, the system can implement any other methods or techniques to collect patient pain data and to serve physical therapy exercise data to the patient in Blocks S130 and S140.

### 7. Patient-Directed Scan

Blocks S150 and S152 of the method recite: prompting the patient to record a first digital photographic image of the joint in flexion in Block S150 and prompting the patient to record a second digital photographic image of the joint in extension in Block S152. Generally, in Blocks S150 and S152, the patient portal guides the patient in recording 2D or 3D scans of a joint of interest in her body through her mobile computing device, such as at full extension, full flexion, full adduction, full abduction, full internal rotation, and/or full external rotation. The system (e.g., the patient portal or the remote computer system) can then process these scans to calculate a range of motion of the patient's joint of interest in one or more degrees of freedom in Block S160, as described below.

In one implementation, following completion of a set of physical therapy exercises prescribed for a self-directed physical therapy session, the patient portal displays a prompt for the patient to: move her left knee into a maximum flexion position that is still “comfortable” (i.e., physically accessible by the patient, even with some pain); and record a single 2D photographic image of her left knee showing portions of both her upper leg (e.g., thigh) and lower leg (e.g., calf). For example, the patient portal can prompt the patient to: sit on a flat surface, such as a floor or a bed; rotate her left hip outwardly; bend her left knee to a maximum conformable flexion angle with her left buttocks, outer left knee (e.g., at her left lateral collateral ligament), and outer left angle in contact with the flat surface; hold her mobile computing device (e.g., a smartphone) over her left knee and level with the flat surface; and then record a single photographic image with the smartphone. In this example, the patient portal can also access an accelerometer (or other inertial and orientation sensor) within the smartphone to detect the orientation of the smartphone and provide guidance to the patient in leveling the smartphone (e.g., normal to gravity) before recording the image. Additionally or alternatively, the patient portal can record the single photographic image and metadata identifying an orientation of the smartphone at a time the smartphone recorded the single photographic image. The system can then apply post-processing techniques to level the single photographic image. For example, the system can: estimate a vector normal to the ground within the single photographic image based on the metadata; and align the single photographic image with the vector (or crop the single photographic image to align with the vector) to level the single photographic image after recording the single photographic image. In this example, the patient portal can also implement objection recognition or other computer vision techniques to identify the patient's left leg in the field of view of the camera and to automatically capture the image when the left leg is identified within an acceptable distance from the camera, the left leg is in focus, and the mobile computing device is level.

In the foregoing implementation, the patient portal can: access a viewfinder of a camera integrated into the mobile computing device; render the viewfinder on the display of the mobile computing device; overlay outlines of an upper leg, a knee in flexion, and a lower leg—in positions approximating the expected maximum flexion of the patient's left knee at the current post-operative date—on the viewfinder; and prompt the patient to align these outlines with corresponding regions of her left leg shown in the viewfinder prior to recording a 2D photographic image. Upon receipt of this photographic image, the patient portal can label select regions of the photographic image according to leg regions indicated by the upper leg, knee, and lower leg outlines rendered on the viewfinder when the photographic image was recorded. The patient portal can additionally or alternatively: render the photographic image on the display of the mobile computing device; prompt the patient to place labels for an upper leg, a knee, and a lower leg directly onto corresponding regions of the photographic image; and/or confirm locations of these labels automatically placed on the photographic image by the patient portal. The system can then implement these labels in Block S160 described below to extract a flexion angle of the patient's left knee from the image.

In the foregoing example, the patient portal can also prompt the patient to record a photographic image of her left leg that includes her left foot. By identifying the patient's left foot and left knee in the photographic image, the patient portal can scale, keystone, or otherwise adjust the image (e.g., according to a projective transform) such that a distance between the patient's left knee and left foot align to a known or reference distance. The patient portal can thus normalize the photographic image based on features identified in the photographic image.

In another implementation, the patient portal prompts the patient to record a 3D scan of her joint of interest. For example, the patient portal can display a prompt for the patient to: move her left knee into a maximum flexion position that is still comfortable; and record a video through her mobile computing device while moving the mobile computing device around her left knee. During this scan the patient portal can also: track the position of the mobile computing device in real space, such as by sampling inertial sensors integrated into the mobile computing device and implementing dead-reckoning techniques; and then stitch video frames recorded during the scan into a 3D model of the patient's left knee and adjacent regions of the patient's upper and lower left leg based on detected changes in position of the mobile computing device in real space through the scan. For example, the patient portal can implement edge detection techniques to detect the outline of the patient's leg in each video frame and then stitch regions of these video frames representing the patient's left leg into a 3D point cloud of the patient's left leg, as shown in FIGS. 1A and 1B. (Alternatively, the patient portal can upload these video frame and position data to the remote computer system, which can transform these data into a 3D model of the patient's left leg.) The patient portal can additionally or alternatively implement contour tracking, texture detection, keypoint descriptors, and/or any other computer vision technique(s) to distinguish the patient's leg tissue and can match these descriptors across successive video frames in order to stitch these video frames into a 3D labeled model of the patient's left leg.

In one variation, the system can prompt the patient to record a set of images (e.g., a set of static images and/or a video) of the joint of interest from a multitude of camera vantage points. The system can then stitch together the set of images captured from the multitude of camera vantage points to generate a 3D representation of the joint. For example, the system can prompt the patient to record a first graphical representation of a first field of view of a camera at a first location in which the joint lies within the first field of view of the camera. The system can also prompt the patient to record a second graphical representation of a second field of view of the camera at a second location distinct from the first location, the joint within the second field of view of the second camera and the second field of view distinct from the first field of view. The system can then compile (i.e., stitch or otherwise align) the first graphical representation and the second graphical representation to define a 3D representation of the joint.

The patient portal can also implement methods and techniques described above to render a dynamic overlay on the viewfinder in order to guide the patient in properly orienting her mobile computing device to capture necessary visual data throughout the scan, and the patient portal can label regions of the 3D model (e.g., clusters of points in the 3D model) of the patient's left leg according to upper leg, knee, and lower leg regions indicated in this dynamic overlay, such as described above.

In the foregoing implementations, the patient portal can also prompt the patient to record a 2D or 3D (optical) scan of her joint of interest while a reference object is placed nearby. For example, a care provider can provide the patient an index card of known size and geometry (e.g., a 60 mm by 100 mm rectangular card); prior to recording a 2D or 3D scan of her left knee, the patient can place the index card on her left thigh just above her left knee. In this example, a reference feature can be printed on the index card, such as in the form of a barcode (e.g., a QR code); upon receipt of the 2D or 3D scan in Block S150, the patient portal can identify the reference feature in the scan, implement edge detection and/or object recognition techniques to detect the perimeter of the index card encompassing the reference feature, and then scale, keystone, dewarp, or otherwise adjust the image according to the known size and geometry of the index card. The patient portal can then extract range of motion data from this adjusted scan in Block S160. Alternatively, in this implementation, the patient can place a credit card, CD disk, or other object of known size and geometry in the field of view of the mobile computing device's camera when recording a scan of her joint of interest in Blocks 150 and S152.

The patient portal can repeat the foregoing methods and techniques to guide the patient in recording a 2D or 3D scan of her joint of interest in various other maximal positions, such as: in maximum comfortable extension; in maximum comfortable adduction; in maximum comfortable abduction; in maximum comfortable internal rotation; and/or in maximum comfortable external rotation. The patient portal can also label each scan with a joint position—such as flexion, extension, adduction, abduction, or rotation—according to a joint position prompt served to the patient before recordation of a scan.

However, the patient portal can implement any other method or technique to guide the patient in collecting a 2D or 3D scan of her joint of interest in Blocks S150 and/or S152 and to prepare this scan for extraction of joint angle in Block S160.

### 7.1 Baseline Scan

In one variation, the system interfaces with the patient or with a care provider to collect a baseline scan of the patient's joint of interest and labels for various features represented in the baseline scan; the system can then project labels from the baseline scan onto post-exercise scans to inform identification of regions of the patient's body recorded in these scans.

In one implementation, a care provider affiliated with the patient interfaces with a separate computing device to generate a baseline scan of the patient's joint of interest prior to the patient's surgery or prior to the discharge of the patient from a hospital. For example, while the patient occupies a room in a hospital (e.g., before or after a surgery), a nurse or technician can scan the patient's joint of interest with a dedicated scanning device, such as a mobile computing device including both a color camera and a distance sensor or an MRI or X-ray machine. The system can then retrieve these scan data to generate a 3D baseline model of the patient's joint of interest and load this 3D baseline model into the patient's user account. In particular, in this implementation, the system can interface with a dedicated computing device manipulated or controlled by a care provider trained in scanning joints of interest in order to generate a 2D or 3D baseline scan containing higher-resolution data and/or exhibiting a higher likelihood of proper completion compared to a 2D or 3D scan performed by the patient on a personal mobile computing device.

In this implementation, the system can also prompt the care provider to label various regions, such as described above. For example, the system can interface with the care provider—who may be professionally trained in building and labeling baseline scans of joints of interest—through the care provider portal to label: the patient's upper leg; the patient's knee; the patient's lower leg; a scar on the patient's knee resulting from the recent total knee replacement surgery; and/or large moles or freckles around the patient's knee (which may function as optical fiducials for projecting labels from the baseline scan onto scans later recorded by the patient in Block S150); etc. in the baseline scan. In particular, the system can leverage the care provider's knowledge of which reference types are useful to the system to label, how many references to label in the baseline scan, and how to correctly label tissues and reference features to achieve a sufficiently accurate and properly labeled baseline scan of the patient's joint of interest.

Alternatively, the system can implement similar methods and techniques to guide the patient in recording and labeling a baseline scan following the surgery, such as before or after a first physical therapy session. However, the system can implement any other methods or techniques to generate a labeled baseline scan.

### 8. Range of Motion Detection

Block S160 of the method recites extracting a first angular position of the joint from the first digital photographic image. Generally, in Block S160, the system can transform a scan of the patient's joint of interest in a known position type (e.g., full comfortable extension or full comfortable flexion) into a maximum comfortable angle of the joint for this position type.

In one example shown in FIGS. 1A and 1B, the system: implements edge detection or other computer vision techniques to identify a region of a scan that represents the patient's left leg; identifies distinct regions of the patient's left leg corresponding to the upper leg, knee, and lower leg based on labels placed directly into the scan by the patient, as described above; predicts a position of the patient's left femur and a position of the patient's left tibia within the scan based on a human leg model and the labeled location of the patient's left knee; projects a first vector along the predicted position of the patient's femur and intersecting the labeled location of the patient's left knee; projects a second vector along the predicted position of the patient's tibia and intersecting the first vector at the labeled location of the patient's left knee; and calculates an angle between the first and second vectors to determine the maximum joint angle of the patient's joint of interest in the position type represented in the scan. Therefore, in this example, the system can implement segmentation techniques to estimate an anatomical structure of the patient around the joint of interest from labeled regions, reference features, and/or tissue geometry represented in the scan.

In the variation described above in which the system generates a baseline scan, the system can project labels from the baseline scan onto a post-exercise scan by: implementing pattern matching or other computer vision techniques to identify reference features in the post-exercise scan that match reference features in the baseline scan; align the baseline scan to the post-exercise scan by aligning like features in the baseline and post-exercise scans; and then writing labels (e.g., upper leg, lower leg, and knee labels) from the baseline scan onto patient tissue represented in the post-exercise scan. The system can then implement the foregoing methods and techniques to extract a joint angle from the post-exercise scan.

The system can repeat this process for each other scan recorded by the patient at her mobile computing device during a physical therapy session in order to aggregate maximum comfortable joint angles for each of various positions relevant to the joint of interest. The system can also compile joint angle pairs into ranges of motion of the joint of interest in one degree of freedom. For example, for a total knee replacement, the system can extract maximum comfortable joint angles for both extension and flexion from two post-exercise scans and then subtract the extension joint angle from the flexion joint angle to calculate range of motion of the knee in a first degree of freedom. However, for a total hip replacement, the system can extract maximum comfortable joint angles for each of extension, flexion, adduction, abduction, internal rotation, and external rotation from six separate scans completed by the patient following one physical therapy session. In this example, the system can then: subtract the extension joint angle from the flexion joint angle to calculate range of motion of the hip in a first degree of freedom; subtract the adduction joint angle from the abduction joint angle to calculate range of motion of the hip in a second degree of freedom; and subtract the internal rotation joint angle from the external rotation joint angle to calculate range of motion of the hip in a third degree of freedom.

However, the system can implement any other method or technique to extract a joint angle from a scan and to merge joints angles extracted from two complementary scans into a range of motion of the joint of interest to one degree of freedom.

### 9. Real-Time Scanning and Range of Motion Detection

In one variation, the system calculates maximum conformable angles of the joint of interest for one or more degrees of freedom in real-time during a physical therapy exercise performed by the patient. For example, the patient can place her mobile computing device on a stand within a room and then perform a prescribed physical therapy exercise in front of the mobile computing device; during the physical therapy exercise, the patient portal executing on the mobile computing device can record a video stream of the patient and then implement methods and techniques similar to those described above (e.g., person detection, human silhouette extraction, and segmentation, etc.) in real-time to extract joint angles and range of motion data from the video stream. In this example, the patient portal can also project labeled regions and/or reference features from the baseline scan onto the video stream to identify and track the joint of interest throughout the physical therapy exercise. Therefore, in this implementation, the system can implement segmentation techniques to estimate an anatomical structure of the patient around the joint of interest from motion captured in the video stream.

### 10. Supplementary Contact-Based Scans

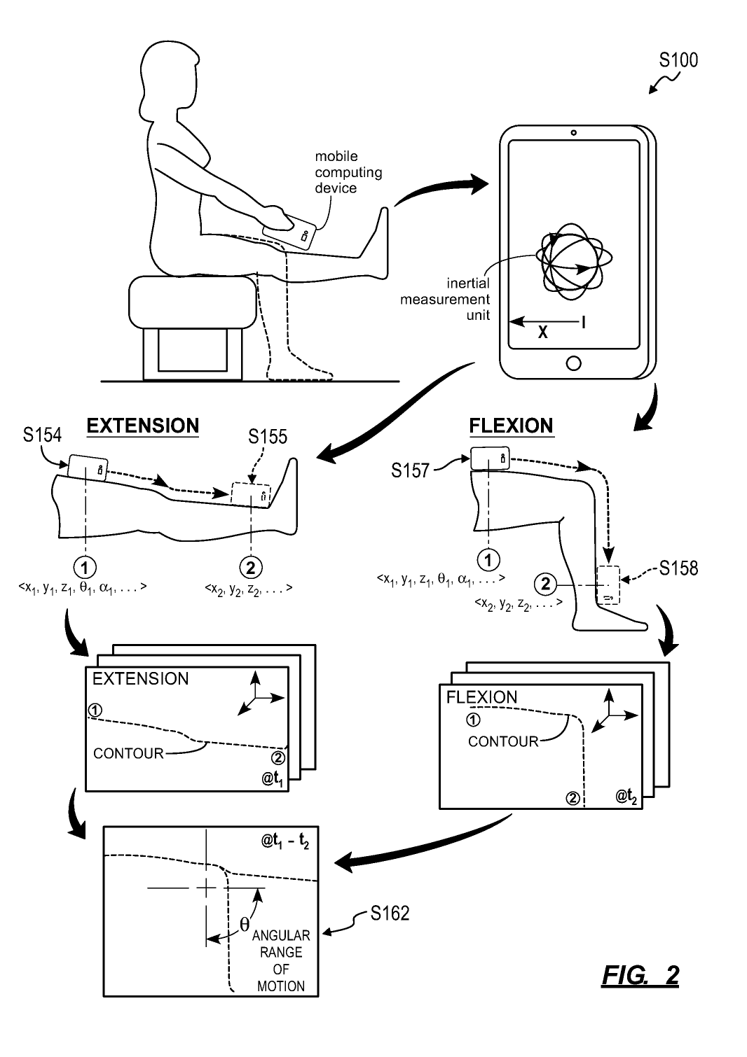

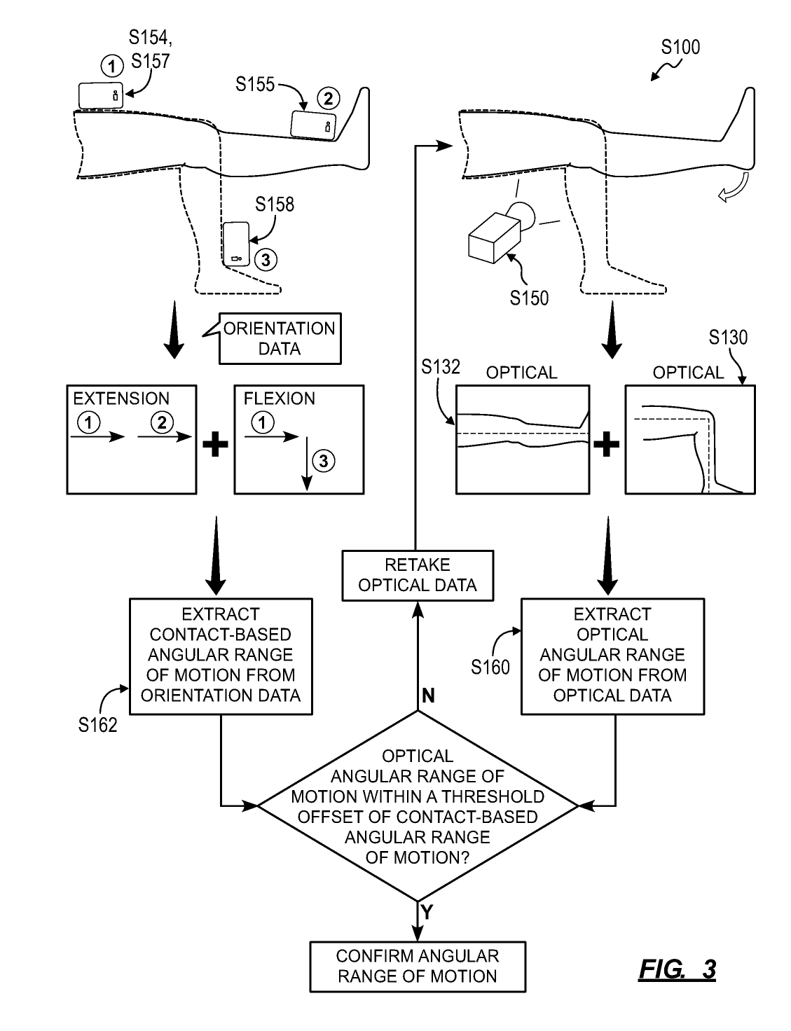

One variation of the method S100 shown in FIGS. 2 and 3 includes, at the mobile computing device during the first physical therapy session: prompting the patient to position the mobile computing device at a first position on a first side of the joint in extension in Block S154; prompting the patient to position the mobile computing device at a second position on a second side of the joint in extension, the second side opposite the joint from the first side of the joint in Block S155; prompting the patient to position the mobile computing device at a third position on the first side of the joint in flexion in Block S157; and prompting the patient to position the mobile computing device at a fourth position on the second side of the joint in flexion in Block S158. Generally, in Blocks S154, S155, S157, and S158, the system and/or the patient portal guides the patient in: aligning her mobile computing device to record orientation data of her mobile computing device (and/or another orientation sensor) proximal locations of her body surrounding the joint of interest. The system can then process the orientation data to generate a 2D and/or 3D contour map of the joint of interest, such as at full extension, full flexion, full adduction, full abduction, full internal rotation, and/or full external rotation. The system (e.g., the patient portal or the remote computer system) can then process this orientation data and/or the contour map to calculate a range of motion of the patient's joint of interest in one or more degrees of freedom in Block S162 described below.

In particular, the patient portal can prompt the patient to align an edge of the mobile computing device with a surface (e.g., skin) of the patient's body proximal the joint of interest (e.g., a thigh portion of the leg proximal a knee joint). In response to confirmation of alignment between the edge of the mobile computing device and the surface of the patient's body, the mobile computing device can then read acceleration values of the mobile computing device along three axes from the IMU.

Based on the acceleration, the mobile computing device can calculate a direction of gravity (or “earth normal) relative to the mobile computing device. Based on the acceleration of the mobile computing device and the direction of gravity, the mobile computing device can determine an orientation of the mobile computing device. Given a known approximate placement of the mobile computing device (e.g., based on placement instructions presented to the user via the mobile computing device), the system can predict an edge of the mobile computing device contacting the patient's skin (e.g., contacting the patient's thigh). The system can calculate an angle of the edge relative to gravity based on the orientation of the mobile computing device and the known edge of the mobile computing device. The system can then store the angle of the edge as representing an angle of a surface of the patient contacting the edge of the mobile computing device (e.g., an angle of the thigh).

In particular, based on a known location of the IMU within a housing of the mobile computing device (and/or a known offset between the IMU and a defined reference point, such as a corner and/or a centroid of the mobile computing device), the system can calculate an orientation of the mobile computing device (e.g., relative to the IMU or to the reference point) based on the angle of the IMU recorded when the mobile computing device was aligned with the region of the patient's body. The system can then estimate orientation and position of a plane approximating an intersection between the edge of the mobile computing device and the surface of the patient's body.

In one implementation, the patient portal can prompt the patient to position the mobile computing device at a first position—contacting or juxtaposed with the patient's skin—adjacent the joint of interest (e.g., in extension, flexion, abduction, adduction, etc.). The patient may confirm alignment of the mobile computing device with the first position through the patient portal. Then the system can detect and record an orientation of the mobile computing device to approximate orientation of a contact plane between the mobile computing device and a surface of the patient's body at the first position. For example, the patient portal can prompt the patient to locate an edge of the mobile computing device (e.g., a back surface) adjacent a thigh portion of a patient's leg (i.e., above the patient's knee). The patient portal can also prompt the patient to select an icon to confirm when the mobile computing device is in position. Upon confirmation, the system can record accelerometer, compass sensor, and/or gyroscope sensor data to generate an orientation vector representing the orientation and position of the mobile computing device. The system can then estimate, from this orientation vector, orientation of a tangential plane representing a contact point between the envelope of the patient's leg at the thigh portion and the back surface of the mobile computing device.

Furthermore, the patient portal can prompt the patient to relocate the mobile computing device to a second position on an opposite side of the joint from the first position. The patient may confirm alignment of the mobile computing device with the second position through the patient portal and the system can detect and record a second orientation of the mobile computing device to approximate orientation of a contact plane between the mobile computing device and a surface of the patient's body at the second position. In the foregoing example, the patient portal can prompt the patient to locate the edge of the mobile computing device adjacent a tibia portion of a patient's leg (i.e., below the patient's knee). The patient portal can prompt the patient to select the icon to confirm when the mobile computing device is in position. Upon confirmation, the system can record accelerometer, compass sensor, and/or gyroscope sensor data to generate an orientation vector representing the orientation and position of the mobile computing device. The system can then estimate orientation of a tangential plane representing a contact point between the envelope of the patient's leg at the tibia portion and the back surface of the mobile computing device. As described below, the system can then extract an angle (e.g., of extension and/or flexion) between the tangential plane representing the contact point at the thigh portion and tangential plane representing a contact point at the tibia portion.

In one implementation, the system can prompt the patient to sweep the mobile computing device along the patient's body from a first position on a first side of the joint over the joint to a second position on a second side of the joint (e.g., in extension and/or flexion). Throughout the sweep, the mobile computing device can record orientation data of the mobile computing device (as described above) defined while sweeping the mobile computing device over the joint of interest. The system can integrate values of the orientation data over the sweep to extract a trajectory of the mobile computing device throughout the sweep. The system can then transform the calculated trajectory of the mobile computing device to estimate a contour of a surface of the patient contacted during the sweep along the trajectory. As described below, the system can extract an angle (e.g., of extension and/or of flexion) defined by the contour of the surface.

The patient portal can repeat the foregoing methods and techniques of this variation to guide the patient in recording a scan of her joint of interest in various other maximal positions, such as: in maximum comfortable extension; in maximum comfortable adduction; in maximum comfortable abduction; in maximum comfortable internal rotation; and/or in maximum comfortable external rotation. The patient portal can also label each scan with a joint position—such as flexion, extension, adduction, abduction, or rotation—according to a joint position prompt served to the patient before recordation of a scan.

However, the system can prompt the patient to scan the joint of interest and areas of the patient's body surrounding the joint of interest through any other suitable method and/or technique.

### 10.1 Range of Motion Detection

Block S162 of the method recites extracting a first angular range of motion of the joint as a difference between an angle of extension and an angle of flexion, the angle of extension defined between the first orientation and the second orientation, the angle of flexion defined between the third orientation and fourth orientation. Generally, the system can transform position and orientation data of a contact point between the envelope of the patient and an edge of the mobile computing device scanned in Blocks S157 and S159 to estimate a maximum comfortable angle of the joint (e.g., in extension and/or in flexion).

As described above, the system: correlates orientation data recorded during a sweep of the edge of an IMU over the joint to estimate a contour of the joint and areas surrounding the joint (e.g., the upper leg, knee, and lower leg). Based on labels applied to the optical scan, a baseline scan, and/or to a generic leg model, as described above, the system can predict a position of the patient's femur and a position of the patient's tibia within the contour. The system can then: project a first vector along the predicted position of the patient's femur and intersecting the labeled location of the patient's left knee; project a second vector along the predicted position of the patient's tibia and intersecting the first vector at the labeled location of the patient's left knee; and calculate an angle between the first and second vectors to determine the maximum joint angle of the patient's joint of interest in the position type represented in the scan. Therefore, in this example, the system can implement segmentation techniques to estimate an anatomical structure of the patient around the joint of interest.

The system can repeat this process for each other scan recorded by the patient at her mobile computing device during a physical therapy session in order to aggregate maximum comfortable joint angles for each of various positions relevant to the joint of interest. The system can also compile joint angle pairs into ranges of motion of the joint of interest in one degree of freedom. For example, for a total knee replacement, the system can extract maximum comfortable joint angles for both extension and flexion from two post-exercise scans and then subtract the extension joint angle from the flexion joint angle to calculate range of motion of the knee in a first degree of freedom.

However, the system can implement any other method or technique to extract a joint angle from a contact-based scan and to merge joint angles extracted from two complementary scans into an angular range of motion of the joint of interest.

### 11. Confidence and Confirmation

In the foregoing variation, the system can merge contact-based scans of the joint with optical scans to confirm and improve confidence that an envelope extracted from the digital photographic images through edge detection or other computer vision techniques identifies the joint of interest as shown in FIG. 3. In this variation, the system can compare an angular range of motion extracted from a digital photographic image of a leg to a second angular range of motion extracted from orientation data recorded during a sweep of the mobile computing device along the leg. In response to the second angular range of motion deviating from the first angular range of motion by less than a threshold deviation error, the system can: project a contour of the leg—extracted from the orientation data—onto a digital photographic image of the joint in a similar position to a position of the joint detected in the contour (e.g., in extension). In response to detecting alignment (or approximate alignment) between the contour and the image of the joint, the system can: increase confidence that the envelope identified in the digital photographic image of the joint corresponds to a leg; increase confidence that the contour of the leg extracted from orientation data corresponds to a contour of a leg; and confirm the first angular range of motion. Therefore, the system can compile contact-based data and optical data to automatically label the optical (photographic) data—and confirm accuracy of labelled optical data—without manual confirmation from a care provider through redundant optical and contact-based data collected by the patient during a training session.

Furthermore, the system can implement the foregoing methods and variations to extract substantially real-time range of motion data from contact-based data to provide prompt feedback to a patient about her recovery progress. The system can also record photographic images of the joint of interest and present these photographic images to the care provider, who may manually extract range of motion information and other metrics to evaluate the patient's progress.

However, in response to the second angular range of motion deviating from the first angular range of motion by more than the threshold deviation error, the system can: flag the angular range of motion extracted from the digital photographic image; prompt the patient to record an additional digital photographic image of the joint (e.g., in extension and/or flexion); and extract a second angular range of motion of the joint from the additional digital photographic image. The system can then compile the second angular range of motion of the joint into a notification as described below.

However, the system can merge orientation data and optical scans to confirm labels, identified features, and/or extracted angles identified in these orientation data and optical scans and improve confidence in accuracy of these labels, identified features, and/or extracted angles in any other suitable way.

### 12. Swelling

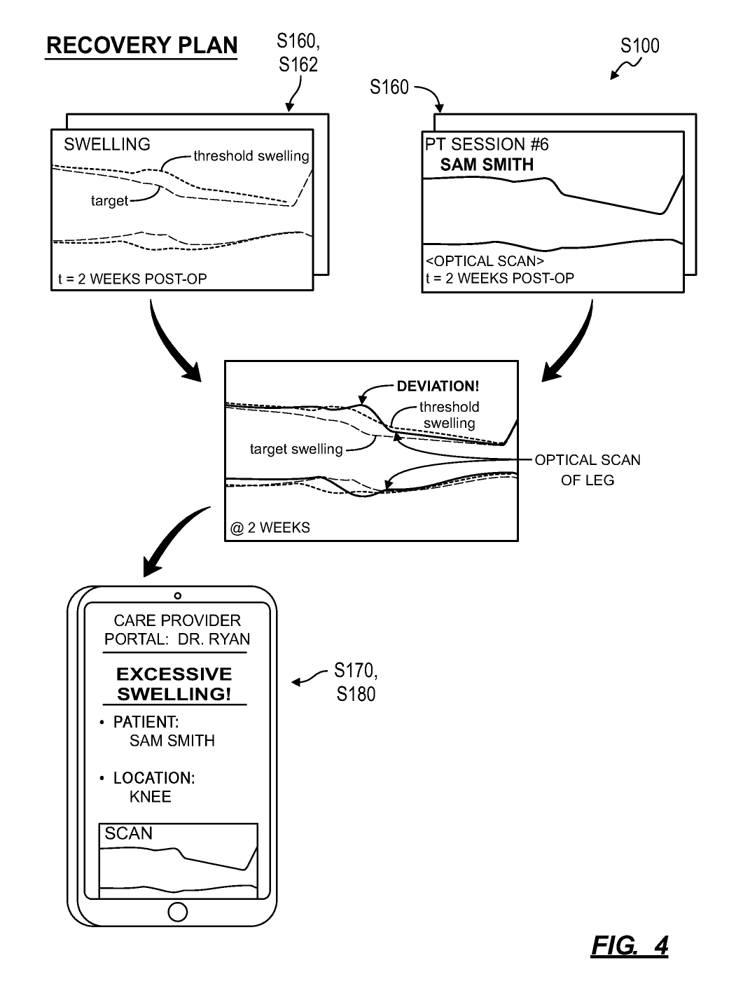

In one variation shown in FIG. 4, the system can: extract a contour of the joint of interest (e.g., from a digital photographic image of the joint by implementing edge detection and/or other computer vision techniques and/or based on orientation data collected by the mobile computing device during a sweep across the joint of interest); and, from the contour of the joint of interest, extract swelling data of tissue surrounding the joint.

In one implementation, the system can access a target swelling threshold and a maximum swelling threshold specified within the recovery plan for the patient for the current number of days post-operation and/or post-commencement of a physical therapy program. The system can compile the target swelling threshold and the maximum swelling threshold with a human model of the joint to generate a target model contour of the joint when swollen to the target swelling threshold and a maximum model contour of the joint when swollen to the maximum swelling threshold. The system can then compare a contour extracted from scans of the patient's joint (e.g., a knee)—as described above—to the target model contour and/or the maximum model contour of the joint. In response to the swelling data extracted from the scans exceeding the maximum swelling threshold (i.e., the contour extracted from scans of the patient's joint deviating from the maximum model contour of the joint), the system can compile the swelling data into a notification transmitted to a care provider and selectively prompt the care provider to address the swelling as described below.

In one variation, the system can access a target change in swelling within the recovery plan for the patient for a particular number of days post-operation and/or post-commencement of the physical therapy program. In this variation, the system can extract trends in swelling from scans of the joint and compare the trends to the target change in swelling (e.g., increases and/or decreases). In response to deviation between the target change in swelling and a current trend in swelling extracted from the scans, the system can compile the swelling data into the notification transmitted to the care provided. For example, at a particular stage of recovery, the recovery plan can indicate increases in swelling are unacceptable and indicate infection. Therefore, in response to detecting an increase in swelling, the system can flag the swelling data and notify a care provider of the increase in swelling. In another example, at another stage of recovery, the recovery plan can define a target rate of decrease in swelling. In response to detecting a decrease in swelling corresponding to the target rate of decrease, the system can flag the patient as compliant with the recovery plan.

However, the system can extract swelling data of the joint—either absolute swelling data and/or changes in swelling over a period of time—from scans of the joint through any other method and/or technique, such as edge detection, template matching, etc.

### 13. Risk Detection

Block S170 of the method recites, in response to the first angular position of the joint deviating from the recovery plan by more than a threshold deviation, compiling the first angular range of motion of the joint and an identifier of the patient into a notification in Block S170; and Block S180 of the method recites serving the notification to a care provider. Generally, the system can predict increased risk to the patient based on differences between the measured range of motion (or discrete joint angles) in the joint of interest at one or more instances in time and a target range of motion (or discrete target joint angles) specified in the recovery plan for corresponding instances in time. In Blocks S170 and S180, the system can compile relevant information regarding the patient's recovery (e.g., the patient's pain level, the measured range of the motion of the joint of interest, and the target range of motion for the joint of interest, etc.) into a notification and then communicate the notification to a care provider affiliated with the patient. The system can thus inform the care provider generally of the patient's condition and prompt the care provider to consider intervening in the patient's recovery when a significant risk to the patient is detected based on quantitative data collected from the patient and quantitative targets set for the patient's recovery.

In one implementation, upon calculating a range of motion in a particular degree of freedom during a current physical therapy session, the system immediately compares this singular range of motion value to a singular target range of motion and corresponding tolerance value specified in the recovery plan for the particular degree of freedom for the current number of days post-operation. In this implementation, the system flags the patient for care provider review if this singular range of motion value differs from (e.g., is less than) the singular target range of motion by more than the corresponding tolerance value. The system can therefore flag the patient for care provider review if the measured range of motion in one degree of freedom in the joint of interest exceeds the target range of motion by more than a corresponding high tolerance and if the measured range of motion in this degree of freedom falls below the target range of motion by more than a corresponding low tolerance specified in the recovery plan for the current number of days since the patient's surgery.

In the foregoing implementation, the system can also calculate a risk score for the patient. For example, the system can calculate a risk score—for the patient during the current physical therapy session—that is proportional to: a number of degrees of freedom in the joint of interest for which ranges of motion have not fallen within corresponding tolerance bands specified in the recovery plan; and/or a magnitude of differences between actual ranges of motion in each specified degree of freedom in the joint of interest and corresponding target ranges of motion specified in the recovery plan. In this example, the system can then flag the patient for care provider review if the risk score exceeds a threshold score.

In another implementation, the system can: calculate a trend (e.g., a parametric time-based trendline, a time-series analysis generated with a predictive model) in the range of motion of the joint of interest in a particular degree of freedom over time (e.g., since a first post-operative physical therapy session); extrapolate range of motion of the joint into the future based on this trend; and compare this extrapolated trend to target range of motion values specified in the recovery plan to predict whether and when the range of motion of the joint of interest in this degree of freedom will differ from the target range of motion values by more than the specified tolerance. Thus, if the extrapolated trend suggests such excessive deviation in the joint of interest in the future and/or if such excessive deviation is predicted to occur at the joint of interest within a threshold period of time (e.g., within one week), the system can flag the patient for care provider review.

The system can additionally or alternatively implement the foregoing methods and techniques to compare discrete maximum comfortable joint angles (e.g., in flexible extension, adduction, etc.) to corresponding target ranges of motion and associated tolerance values specified in the recovery plan for the current number of days post-operation, as shown in FIGS. 1A and 1B. The system can thus flag the patient for care provider review if one or more of these discrete maximum comfortable joint angles currently differs from its corresponding target range of motion by more than the associated tolerance value or is predicted to differ from its corresponding target range of motion by more than the associated tolerance value in the future.

The system can also repeat the foregoing methods and techniques for each distinct range of motion specified in the recovery plan; and the system can flag the patient for care provider review if the range of motion or discrete joint angles in a minimum subset of these degrees of freedom currently or is predicted to deviate significantly from its corresponding target range of motion value.

### 13.1 Other Triggers

Additionally or alternatively, the system can also flag the patient responsive to various other triggers, such as if the patient has missed more than a threshold number of (e.g., two) self-directed physical therapy sessions, if the patient has entered relatively high or anomalously-high perceived pain values in Block S130, if swelling (or change in swelling) exceeds a maximum swelling threshold, or if relative perceived pain values entered by the patient are trending upwardly or not trending downwardly by a rate specified by the recovery plan.

Furthermore, the system can also flag the patient responsive to trends in patient range of motion, swelling, pain, etc. values extracted during the self-directed physical therapy sessions. For example, the system can access historical range of motion data of the joint and append the historical range of motion data with a latest angular range of motion value extracted from a scan as described above. In this example, the system can identify and/or extract a velocity (i.e., a trend or rate) of change in range of motion of the joint during recovery from the historical range of motion data. In response to the velocity falling outside a threshold tolerance for velocity of change in range of motion specified in the recovery plan, the system can flag the patient for review by a care provider. In this example, if the patient's range of motion of the joint increases slowly post-operatively, thereby improving at a slow velocity, the system can flag the patient as “progressing slowly” and notify a care provider to review the patient's physical therapy program and/or recovery plan. However, if the patient's range of motion of the joint changes quickly post-operatively, thereby improving at a higher velocity, the system can flag the patient as “progressing quickly” and also notify a care provider to review the patient's physical therapy program and/or recovery plan to assist the patient in avoiding recurring injuries. Furthermore, if the patient's range of motion of the joint declines (i.e., a negative velocity slope), the system can flag the patient as “regressing” and notify a care provider, such as a physician, to contact the patient and address the declining range of motion promptly.

In a similar example, the system can identify or extract an acceleration (or rate of change of the velocity during recovery) from the historical range of motion data. In response to the acceleration falling outside a threshold tolerance for acceleration of change in range of motion specified in the recovery plan, the system can flag the patient for review by a care provider. In this example, if the rate at which the patient's range of motion of the joint increases slowly post-operatively, thereby improving at a slow acceleration, the system can flag the patient as “progressing slowly” and notify a care provider to review the patient's physical therapy program and/or recovery plan.

### 14. Notifications

Once the system has flagged the patient for care provider review, the system can package patient data for immediate access by the care provider, such as in a visualization presented to the care provider within a dashboard with the care provider portal. For example, the system can generate a graph containing the measured range of motion in the joint of interest in one degree of freedom, a corresponding target range of motion value defined in the recovery plan, and/or a tolerance band defined in the recovery plan plotted versus time (e.g., days) since the patient's surgery or since commencement of the physical therapy plan. The system can also represent perceived pain levels entered by the patient and/or physical therapy exercises completed by the patient over time in this graph. The system can generate one such graph per degree of freedom for the joint of interest or compile these plots into a single graph. Furthermore, the system can pair these graphs with other patient data, such as physical therapy exercises completed by the patient, the date and type of the patient's surgery, and/or the patient's name, phone number or point of contact, age, mobility, weight, body-mass index, and/or other medical condition or complications, etc. For example, in Block S170, the system can aggregate these graphs and other patient data into a single electronic notification, such as in the form of an email, text message, or push notification accessible through the care provider portal. Alternatively, the system can: generate a notification indicating that review of a patient is requested at the care provider portal; push this notification to the care provider's mobile computing device; and load these patient data into the care provider portal for review by the care provider following secured login into the care provider portal.

The system can then push the electronic notification to the care provider in Block S180 for manual review, as shown in FIGS. 1A and 1B. For example, by presenting the care provider with a visual, graphical representation of the patient's perceived pain level, range of motion in one or more degrees of freedom, and/or completed physical therapy exercises over time, the system can enable the care provider to quickly ascertain the patient's recovery risk; by providing other patient-related data, the system can enable the care provider to quickly re-familiarize herself with the patient. The care provider can consider these data: to determine that the patient is recovering adequately and to instead revise her assigned recovery plan; to determine that the patient is exhibiting low risk sufficient to justify a change to the prescribed physical therapy program and to adjust the physical therapy program accordingly; to determine that the patient is exhibiting moderate risk sufficient to justify a meeting with a doctor or to justify transition to physical therapy sessions directed in-person by a physical therapist; and/or determine that the patient is exhibiting high risk sufficient to justify a second corrective surgery; etc.

### 14.1 Patient Prioritization

In one implementation, the system can selectively prioritize a particular patient over a population of patients at a similar stage of recovery; and transmit a notification to the care provider indicating the priority of the particular patient. Generally, the system can prioritize patients at high risk for recurring injury and/or other complications, such as infection, limited mobility, loss of range of motion, high pain levels, opioid addiction, etc., over lower-risk patients.

As described above, the system can assign a priority level based on deviation of a calculated range of motion (i.e., extracted from optical scans and/or contact-based scans) from a target range of motion prescribed within the recovery plan. In one implementation, in response to the first angular range of motion of the joint deviating from the recovery plan by more than the threshold deviation, the system can assign a high priority level to the patient. However, in response to the first angular range of motion of the joint deviating from the recovery plan by less than the threshold deviation, the system can assign priority levels based on urgency of response to the symptom captured by the system. For example, in response to the velocity falling outside the threshold tolerance for velocity, the system can assign a medium priority level to the patient as the patient's range of motion is changing faster or slower than a target pace of change in range of motion prescribed within the recovery plan. In response to the velocity falling within the threshold tolerance for velocity, the system can assign a low priority level to the patient. Similarly, in response to the rate of change of the velocity (i.e., acceleration) falling outside a threshold tolerance for rate of change of the velocity of change in range of motion, the system can assign a medium priority level.

However, the system can assign priority levels and/or dictate priority of a patient in any other suitable way. For example, the system can assign priority levels based on risk of infection, swelling data, number of exercises assigned to the patient in the physical therapy program, a number of missed or skipped physical therapy sessions, etc.

After assigning a priority level to the patient, the system can then serve to the care provider a list of patients affiliated with the care provider ordered according to the priority level. Alternatively, the system can selectively serve an alert to the care provider when a patient's priority level exceeds a “medium” (or other threshold) level and/or whether the patient's priority level changes. Therefore, the system can assist the care provider in prioritizing care for high-risk patients.

### 14.2 Care Provider Selection

In another implementation, the system can selectively serve notifications to a particular care provider of the patient, such as a doctor, a physical therapist, an engineer, and/or a coach, based on range of motion data, pain levels, and/or swelling data recorded by the mobile computing device. Generally, the system can select a particular care provider from a set of care providers available to the patient based on a risk level of the patient, a priority level of the patient, and/or other metrics indicating instant needs of the patient.

For example, the system can select a physician from a set of care providers affiliated with the patient in response to detecting the high priority level assigned to the patient or a large deviation in range of motion from a target range of motion prescribed in the recovery plan. The system can similarly select a physical therapist from the set of care providers in response to detecting the medium priority level assigned to the patient and/or a deviation in pain level from the target pain level prescribed in the recovery plan. Additionally, the system can select a recovery coach (e.g., a nurse) from the set of care providers in response to detecting the low priority level assigned to the patient. Therefore, the system can selectively prompt an expensive care provider—like a physician—to respond to high-priority or high-risk patients exclusively to avoid incurring excessive costs for recovery by dispatching a care provider according to her expertise (and cost of her time) and ensuring expertise proportional to the risk and/or priority of the patient.

Following selection of an appropriate care provider, the system can selectively transmit the notification (exclusively) to the appropriate care provider to: avoid unnecessarily notifying an expensive or over-qualified care provider; avoid dispatching a care provider ill-equipped to address a patient's issues; and assist care providers in identifying patients to assist imminently. However, the system can selectively notify care providers of the patient in any other suitable way.

### 15. Care Provider Response and Plan Adaptation

In the foregoing implementation, the system can collect feedback entered by the care provider to adjust the recovery plan assigned to the patient and to push this update to the patient's user account. The system can also automatically update a corresponding recovery plan template such that recovery plans assigned to similar patients (of the same hospital, surgical group, health insurance company, physical therapy group, etc.) following the same surgery type in the future reflect this adjustment by the care provider, as shown in FIGS. 1A and 1B. Similarly, if the care provider confirms that the original recovery plan for the patient was correct or adequate, the system can reinforce the original recovery plan for future patients of similar demographic with the same prescribed surgery type. The system can therefore implement supervised machine learning techniques to automatically adjust recovery plan templates, as described above, based on care provider feedback in order to improve prediction of patient risk and to improve accuracy in selectively notifying care providers of at-risk patients.

Furthermore, the system can collect feedback entered by the care provider to adjust the physical therapy program assigned to the patient and to push this update to the patient's user account. The system can also automatically update a corresponding physical therapy program template such that physical therapy programs assigned to similar patients following the same surgery type in the future reflect this adjustment by the care provider. If the care provider confirms that the original physical therapy program for the patient was correct or adequate, the system can also reinforce the original physical therapy program for future patients of a similar demographic with the same prescribed surgery type. The system can therefore implement supervised machine learning techniques to automatically adjust physical therapy program templates, as described above, based on care provider feedback in order to improve types and/or intensities of physical therapy exercises prescribed to patients following surgical operations in the future.

The systems and methods described herein can be embodied and/or implemented at least in part as a machine configured to receive a computer-readable medium storing computer-readable instructions. The instructions can be executed by computer-executable components integrated with the application, applet, host, server, network, website, communication service, communication interface, hardware/firmware/software elements of a user computer or mobile device, wristband, smartphone, or any suitable combination thereof. Other systems and methods of the embodiment can be embodied and/or implemented at least in part as a machine configured to receive a computer-readable medium storing computer-readable instructions. The instructions can be executed by computer-executable components integrated by computer-executable components integrated with apparatuses and networks of the type described above. The computer-readable medium can be stored on any suitable computer readable media such as RAMs, ROMs, flash memory, EEPROMs, optical devices (CD or DVD), hard drives, floppy drives, or any suitable device. The computer-executable component can be a processor but any suitable dedicated hardware device can (alternatively or additionally) execute the instructions.

As a person skilled in the art will recognize from the previous detailed description and from the figures and claims, modifications and changes can be made to the embodiments of the invention without departing from the scope of this invention as defined in the following claims.

I claim:

1. A method for tracking patient recovery during a physical therapy program comprising:
assigning a recovery plan to a patient, the recovery plan defining temporal quantitative targets for range of motion of a joint;
at a mobile computing device during a first physical therapy session:
prompting the patient to record a first digital photographic image of the joint in flexion; and
prompting the patient to record a second digital photographic image of the joint in extension;
extracting a first angular range of motion of the joint from the first digital photographic image and the second digital photographic image;
in response to the first angular range of motion of the joint deviating from the recovery plan by more than a threshold deviation, compiling the first angular range of motion of the joint and an identifier of the patient into a notification; and
serving the notification to a computing device associated with a care provider.

2. The method of claim 1:
wherein assigning the recovery plan to a patient comprises assigning the recovery plan to the patient based on a surgery prescribed to the patient, the recovery plan defining temporal quantitative targets for range of motion of the joint addressed in the surgery; and
further comprising:
assigning a physical therapy program to the patient, the physical therapy program defining a set of physical therapy exercises; and
at the mobile computing device during the first physical therapy session, serving a first physical therapy exercise in the physical therapy program to the patient.

3. The method of claim 1, wherein serving the notification to the care provider comprises, in response to the first angular range of motion of the joint deviating from the recovery plan by more than the threshold deviation:
prioritizing the patient over a population of patients at a similar stage of recovery; and
transmitting the notification to the care provider.

4. The method of claim 3:
further comprising:
appending historical range of motion data with the first angular range of motion; and
extracting a velocity of change in range of motion of the joint during recovery from the historical range of motion data;
wherein prioritizing the patient over a population of patients at a similar stage of recovery comprises:
in response to the first angular range of motion of the joint deviating from the recovery plan by more than the threshold deviation, assigning a high priority level to the patient; and
in response to the first angular range of motion of the joint deviating from the recovery plan by less than the threshold deviation and in response to the velocity falling outside a threshold tolerance for velocity, assigning a medium priority level to the patient; and
wherein transmitting the notification to the care provider comprises selectively transmitting a priority level assigned to the patient.

5. The method of claim 4:
further comprising extracting an acceleration of change in range of motion of the joint during recovery from the historical range of motion data;
wherein prioritizing the patient over a population of patients at a similar stage of recovery comprises:
in response to the first angular range of motion of the joint deviating from the recovery plan by less than the threshold deviation and in response to the acceleration falling outside a threshold tolerance of acceleration of change in range of motion of the joint, assigning the medium priority level to the patient; and
in response to the first angular range of motion of the joint deviating from the recovery plan by less than the threshold deviation and in response to the rate of change of the velocity falling within the threshold tolerance of acceleration of change in range of motion of the joint, assigning a low priority level to the patient; and
wherein selectively transmitting a priority level assigned to the patient comprises serving to the care provider a list of patients affiliated with the care provider ordered according to the priority level.

6. The method of claim 5, wherein serving the notification to the care provider comprises:
selecting a physician from a set of care providers affiliated with the patient in response to detecting the high priority level assigned to the patient;
selecting a physical therapist from the set of care providers in response to detecting the medium priority level assigned to the patient;
selecting a recovery coach from the set of care providers in response to detecting the low priority level assigned to the patient; and
transmitting the notification to a mobile computing device affiliated with the care provider.

7. The method of claim 1:
further comprising, at the mobile computing device during the first physical therapy session, prompting the patient to enter a first pain level; and
wherein compiling the first angular range of motion of the joint and the identifier of the patient into the notification comprises compiling the first pain level, the first angular range of motion of the joint, and the identifier of the patient into the notification.

8. The method of claim 1, further comprising:
at the mobile computing device during the first physical therapy session:
prompting the patient to position the mobile computing device at a first position on a first side of the joint in extension;
prompting the patient to position the mobile computing device at a second position on a second side of the joint in extension, the second side opposite the joint from the first side of the joint;
prompting the patient to position the mobile computing device at a third position on the first side of the joint in flexion;
prompting the patient to position the mobile computing device at a fourth position on the second side of the joint in flexion;
extracting an angle of extension as a function of the first position and the second position;
extracting an angle of flexion as a function of the third position and the fourth position; and
calculating a second angular range of motion of the joint as a difference between the angle of extension and the angle of flexion.

9. The method of claim 8:
wherein prompting the patient to position the mobile computing device at the first position comprises prompting the patient to sweep the mobile computing device from the first position on the first side of the joint in extension over the joint to the second position on the second side of the joint in extension, the mobile computing device contacting a first section of the patient while sweeping the mobile computing device from the first position to the second position;
further comprising extracting a first contour of the patient corresponding to the first section of the patient contacted by the mobile computing device while sweeping the mobile computing device from the first position to the second position;
wherein prompting the patient to position the mobile computing device at the third position comprises prompting the patient to sweep the mobile computing device from the third position on the first side of the joint in flexion over the joint to the fourth position on the second side of the joint in flexion, the mobile computing device contacting a second section of the patient surrounding the joint while sweeping the mobile computing device from the third position to the fourth position; and
further comprising extracting a second contour of the patient corresponding to the second section of the patient contacted by the mobile computing device while sweeping the mobile computing device from the third position to the fourth position.

10. The method of claim 9, further comprising:
extracting swelling data of tissue surrounding the joint based on the first contour and the second contour;
in response to detecting an increase in swelling over a period, serving a prompt to the care provider to address swelling in the patient;
in response to a decrease in swelling over the period, serving a notification to the patient indicating an acceptable change in swelling.

11. The method of claim 10, further comprising in response to the second angular range of motion deviating from the first angular range of motion by less than a threshold deviation error:
projecting the first contour of the joint in extension on an image of the joint in extension; and
in response to detecting alignment between the first contour and the image of the joint in extension, confirming the first angular range of motion.

12. The method of claim 8:
wherein assigning the recovery plan to a patient comprises defining temporal quantitative targets for range of motion of a leg;
wherein prompting the patient to position the mobile computing device at the first position comprises prompting the patient to align an edge of the mobile computing device with a thigh portion of the leg;
wherein prompting the patient to position the mobile computing device at the second position comprises prompting the patient to align the edge of the mobile computing device with a tibia portion of the leg;
wherein prompting the patient to position the mobile computing device at the third position comprises prompting the patient to align an edge of the mobile computing device with the thigh portion of the leg proximal the first position; and
wherein prompting the patient to position the mobile computing device at the fourth position comprises prompting the patient to align the edge of the mobile computing device with a tibia portion of the leg proximal the second position.

13. The method of claim 8, further comprising, in response to the second angular range of motion deviating from the first angular range of motion by more than a threshold deviation error:
at the mobile computing device:
prompting the patient to record a third digital photographic image of the joint in flexion;
prompting the patient to record a fourth digital photographic image of the joint in extension; and
extracting a third angular range of motion of the joint from the third digital photographic image and the fourth digital photographic image; and
compiling the third angular range of motion into the notification.

14. The method of claim 1, further comprising:
compiling the first angular range of motion with historical range of motion data to define a set of range of motion data identifying changes in range of motion during the physical therapy plan; and
serving the set of range of motion data to a computing device affiliated with the patient.

15. The method of claim 1, wherein prompting the patient to record the first digital photographic image of the joint in flexion comprises recording a three-dimensional image of the joint through a stereo camera integrated into the mobile computing device.

16. A method for tracking patient recovery during a physical therapy program comprising:
assigning a recovery plan to a patient, the recovery plan defining temporal quantitative targets for range of motion of a joint;
at a mobile computing device during a first physical therapy session:
prompting the patient to record a first orientation of the mobile computing device at a first position on a first side of the joint in extension;
prompting the patient to record a second orientation of the mobile computing device at a second position on a second side of the joint in extension, the second side opposite the joint from the first side of the joint;
prompting the patient to record a third orientation of the mobile computing device at a third position on the first side of the joint in flexion;
prompting the patient to record a fourth orientation of the mobile computing device at a fourth position on the second side of the joint in flexion;
extracting a first angular range of motion of the joint as a difference between an angle of extension and an angle of flexion, the angle of extension defined between the first orientation and the second orientation, the angle of flexion defined between the third orientation and fourth orientation;
in response to the first angular range of motion of the joint deviating from the recovery plan by more than a threshold deviation, compiling the first angular range of motion of the joint and an identifier of the patient into a notification; and
serving the notification to a computing device associated with a care provider.

17. The method of claim 16, further comprising:
at the mobile computing device during the first physical therapy session:
prompting the patient to record a first digital photographic image of the joint in flexion; and
prompting the patient to record a second digital photographic image of the joint in extension;
extracting a second angular range of motion of the joint from the first digital photographic image and the second digital photographic image;
in response to the first angular range of motion deviating from the second angular range of motion by more than a threshold offset:
prompting the patient to record a third digital photographic image of the joint in flexion; and
prompting the patient to record a fourth digital photographic image of the joint in extension; and
extracting a third angular range of motion of the joint from the first digital photographic image and the second digital photographic image; and
in response to the first angular range of motion deviating from the second angular range of motion by less than the threshold offset, confirming the first angular range of motion.

18. The method of claim 17:
further comprising, in response to the first angular range of motion deviating from the second angular range of motion by less than the threshold offset, assigning a confidence value to the first angular range of motion; and
wherein compiling the first angular range of motion of the joint and the identifier of the patient into the notification comprises compiling the confidence value into the notification.

19. The method of claim 16:
wherein prompting the patient to record the first orientation of the mobile computing device comprises prompting the patient to sweep the mobile computing device from the first position on the first side of the joint in extension over the joint to the second position on the second side of the joint in extension, the mobile computing device contacting a first section of the patient while sweeping the mobile computing device from the first position to the second position;
wherein prompting the patient to record the third orientation of the mobile computing device comprises prompting the patient to sweep the mobile computing device from the third position on the first side of the joint in flexion over the joint to the fourth position on the second side of the joint in flexion, the mobile computing device contacting a second section of the patient surrounding the joint while sweeping the mobile computing device from the third position to the fourth position; and
further comprising:
extracting a first contour of the patient corresponding to the first section of the patient contacted by the mobile computing device while sweeping the mobile computing device from the first position to the second position;
extracting a second contour of the patient corresponding to the second section of the patient contacted by the mobile computing device while sweeping the mobile computing device from the third position to the fourth position;
extracting swelling data of tissue surrounding the joint based on the first contour and the second contour; and
in response to the swelling data exceeding a maximum swelling threshold, serving a prompt to the care provider to address swelling in the patient.

20. The method of claim 16, wherein serving the notification to the care provider comprises, in response to the first angular range of motion of the joint deviating from the recovery plan by more than the threshold deviation:
prioritizing the patient over a population of patients at a similar stage of recovery; and
transmitting the notification to the care provider selected from a set of care providers based on deviation of the first angular range of motion of the joint from the recovery plan.
